(19) United States
(12) Patent Application Publication
(10) Pub. No.: US 2014/0207053 A1
(43) Pub. Date: Jul. 24, 2014

(54) DEVICE AND METHOD FOR THE PLASMA TREATMENT OF SURFACES AND USE OF A DEVICE

(71) Applicants: Gregor Morfill, Muenchen (DE); Yangfang Li, Garching (DE); Julia Zimmermann, Muenchen (DE); Tetsuji Shimizu, Garching (DE)

(72) Inventors: Gregor Morfill, Muenchen (DE); Yangfang Li, Garching (DE); Julia Zimmermann, Muenchen (DE); Tetsuji Shimizu, Garching (DE)

(73) Assignee: Max-Planck-Gesellschaft zur Foerderung der Wissenschaften e.V., Muenchen (DE)

(21) Appl. No.: 14/115,789

(22) PCT Filed: May 4, 2012
(86) PCT No.: PCT/EP2012/001924

§ 371 Date: Jan. 24, 2014

(30) Foreign Application Priority Data
May 5, 2011 (DE) 10 2011 100 751.6
May 25, 2011 (DE) 10 2011 102 589.1

(51) Int. Cl. A61B 18/04

(52) U.S. Cl. CPC A61B 18/042
USPC 604/24; 604/23

(57) ABSTRACT

The invention relates to a device for the plasma treatment of surfaces (47), in particular of skin, having a housing (3), a plasma source (5) allocated to the housing (3), and at least one spacer (21) that is provided on the housing (3) and/or the plasma source (5) in such a manner that a distance (d) between the plasma source (5) and a surface (47) to be treated can be maintained at least in some regions. The device is characterized in that the spacer (21) is designed to be adjustable in order to vary the distance (d) and that the distance (d) can be varied in such a manner that a preferred plasma chemistry can be selected at the location of the surface (47) to be treated.

The invention concerns an apparatus for plasma treatment of surfaces according to the preamble of claim 1, a method for plasma treatment of surfaces according to the preamble of claim 10 and use of an apparatus for plasma treatment of surfaces according to the preamble of claim 16.

Apparatuses and methods for plasma treatment of surfaces as well as applications of apparatuses for this purpose are known. Typically a plasma is generated by means of a plasma source, which acts on the surface being treated in order to modify it, especially to sterilize, decontaminate and/or disinfect it. A spacer device can then be provided, by means of which a predetermined distance between the plasma source and the surface being treated is maintained. Because of this, it can be ensured, on the one hand, that the distance is sufficient to be able to effectively ignite a plasma and that, on the other hand, an unduly large distance is not chosen so that effective plasma treatment is no longer possible. It has turned out that, depending on the specifically present distance between the plasma source and the surface being treated, specific chemical and/or physical species are preferably present at the treatment location. In this case, depending on the desired plasma treatment, a specific plasma chemistry, i.e., a specific composition or concentration of the species present in the plasma is advantageous. In known apparatuses, methods and applications of known apparatuses the treatment distance, however, is established so that the same plasma chemistry is always present regardless of the actual treatment. Known apparatuses, methods and applications are therefore more suited for certain types of treatment and less for others. Ultimately, it is therefore necessary to keep on hand a separate apparatus for each type of treatment, which is expensive and cumbersome. On the other hand, specific types of treatment can only be conducted with restricted effectiveness, if, for example, only one or only a few apparatuses are available.

It is the therefore the task of the invention to devise an apparatus, a method and application of an apparatus by means of which the plasma chemistry can be selected at the treatment location. Quite different types of treatment can be conducted with the same apparatus on this account. This simplifies plasma treatment, increases its effectiveness and/or reduces the costs.

The task is solved by devising an apparatus with the features of claim 1. This serves for plasma treatment of surfaces, especially skin. It includes a housing, plasma source assigned to the housing and a spacer device, in which the spacer device is provided on the housing and/or the plasma source so that at least in areas a distance can be maintained between the plasma source and the surface being treated. The apparatus is characterized by the fact that the spacer device is designed adjustable so that the distance between the plasma source and the surface being treated, namely the treatment distance can be varied, in which case it can be varied so that a preferred plasma chemistry can be selected at the location of the surface being treated. Variation must therefore be able to be performed in a specific distance range so that the concentration of active species at the treatment location can be adjusted. Consequently, it is possible to carry out a variety of treatment types with the same apparatus in which the plasma chemistry optimal for the corresponding treatment can be selected.

A distance of about 0 to 30 mm, especially about 4 to 20 mm can be set by means of the spacer device. These are typical distances within which the concentration of active species is changed at the treatment location or certain species are present in desired concentration.

An apparatus, in which the spacer device has threading, which meshes with corresponding threading of the apparatus, is preferred. The distance can then be adjusted very simply by pivoting or rotating the spacer device relative to the rest of the apparatus so that a change in treatment distance occurs because of the meshing thread.

An apparatus is also preferred in which the spacer device has at least one snap-in element, which cooperates with at least a corresponding snap-in element of the apparatus in order the define the spacing. Such corresponding snap-in elements are known for hair clippers. In particular, in such a practical example it is possible to adjust the treatment distance by relative movement of the spacer device relative to the remainder of the apparatus, viewed in its longitudinal direction.

The apparatus, especially the spacer device, preferably has a scale, by means of which a desired distance and a preferred plasma chemistry can be set. The scale, for example, can be a distance scale. It is also possible to characterize the plasma chemistry active at a certain distance in the form of pictograms or by indication of the reactive species.

Finally, an apparatus is preferred which has a selection device, by means of which a preferred plasma chemistry can be selected preferably automatically. In particular, the selection device can preferably select a desired type of treatment and adjusts the plasma chemistry and ultimately the treatment distance to it. It can then display the desired distance in a display so that it can be manually adjusted or it can automatically adjust the spacer device preferably via an actuator.

Additional advantageous embodiments are apparent from the dependent claims.

The task is also solved by devising a method for plasma treatment of surfaces, especially skin, with the features of claim 10. The method includes the following steps: an effect mechanism for the surface being treated is selected. The distance from the plasma source to the surface being treated, i.e., the treatment distance is adjusted. The plasma treatment is conducted. The method is therefore applicable to many different types of treatment because an appropriate distance and therefore an appropriate plasma chemistry can always be selected at the treatment location.

A method is preferred in which the distance is set between roughly 0 and 30 mm and preferably between roughly 4 and 20 mm. These are typical distance ranges within which preferred active species or compositions of the plasma active at the treatment location can be selected.

With particular preference selection of the distance occurs by means of a selection device. This preferably permits automatic distance selection, in which the following steps with particular preference are carried out: at least one characteristic value of the surface being treated is recorded. The recorded value is compared with at least one reference value. The recorded value is assigned to at least one reference value. Finally the treatment distance is determined by means of the at least one assigned reference value. A database with reference values is preferably provided so that a treatment requirement of the surface being treated can be established by comparison of the recorded value with the reference values. Preferably these data are linked to a plasma chemistry that is particularly effective with respect to the treatment required in conjunction with the reference values. The plasma chemistry is preferably linked to a distance parameter that corresponds to optimal treatment distance. It is preferably also possible that the distance is calculated with respect to the desired plasma chemistry. As an alternative, the distance parameter can also be linked to the reference values directly.

A method is preferred in which the at least one recorded value is a picture of a surface being treated, which is compared with at least one reference picture. The method is then preferably characterized by the following steps: at least one picture of the surface to be treated is recorded. At least one picture is compared with at least one reference picture. The at least one picture is assigned to at least one reference picture. A plasma chemistry preferably linked to the at least one reference picture is selected. A distance corresponding to the selected plasma chemistry is determined.

Preferably the distance is calculated as a function of the desired plasma chemistry with reference to at least a reaction rate for formation and/or decomposition of the desired species as well as at least a relevant diffusion constant for the plasma. Such calculation is based on the finding that the species formed in the plasma or by the plasma or the reactants have certain diffusion constants for their formation. The plasma chemistry desired at the treatment location is also characterized by reaction rates with which the active species are formed and decomposing it. A distance from the plasma source is attained from the reaction rates on the one hand and the diffusion constants, on the other hand, at which a desired plasma chemistry, i.e., a desired concentration of certain reactive species occurs. Accordingly, the distance can be calculated, if the diffusion constants and reaction rates are known.

Additional advantageous embodiments are apparent from the dependent claims.

The task is finally also solved by devising use of an apparatus with the features of claim 16. An apparatus with a plasma source and a spacer device is used for plasma treatment of surfaces, especially skin, especially an apparatus according to one of the claims 1 to 9. The apparatus is preferably used for treatment of wounds, skin irritation, infections, insect bites, fungal infestation of the feet, especially athlete's foot, acne, herpes, pimples, burns, ear infections, rashes, especially diaper rash, heat blisters, lice infestation, especially the head or body, flea infestation, other infestation by invertebrates, dandruff, sensitive skin, fungal infestation of the nails, psoriasis, fever blisters. It is also or as an alternative used to prevent, eliminate, modify or reduce body odor, for example, foot odor, armpit odor, odor caused by incontinence, odor of the pubic area and/or odor escaping from the body interior. The apparatus is preferably used in a method according to one of the claims 10 to 14.

The invention is further explained below with reference to the drawing. In the drawing:

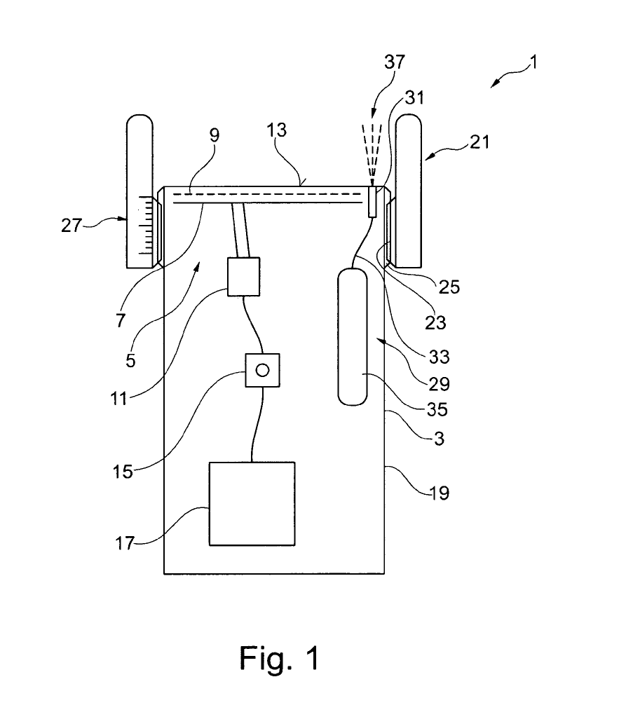

FIG. 1 shows a schematic view of the first practical example of an apparatus for plasma treatment of surfaces;

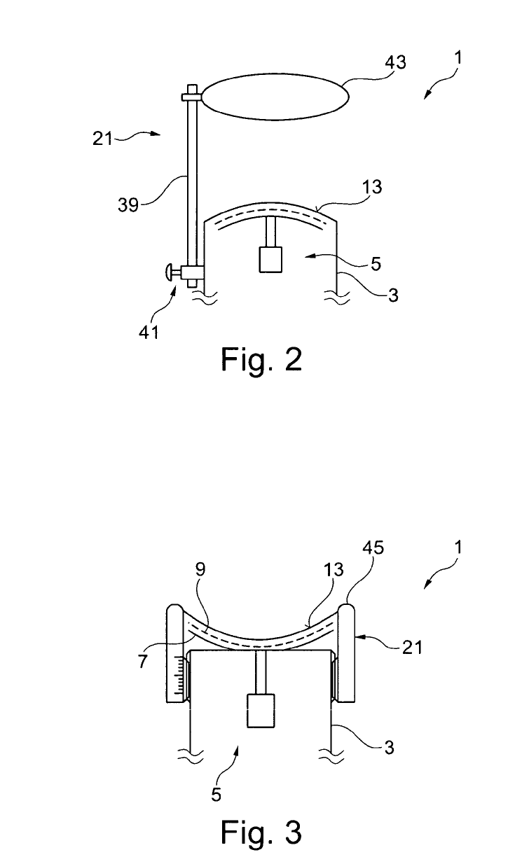

FIG. 2 shows a second practical example of an apparatus;

FIG. 3 shows a third practical example of an apparatus and

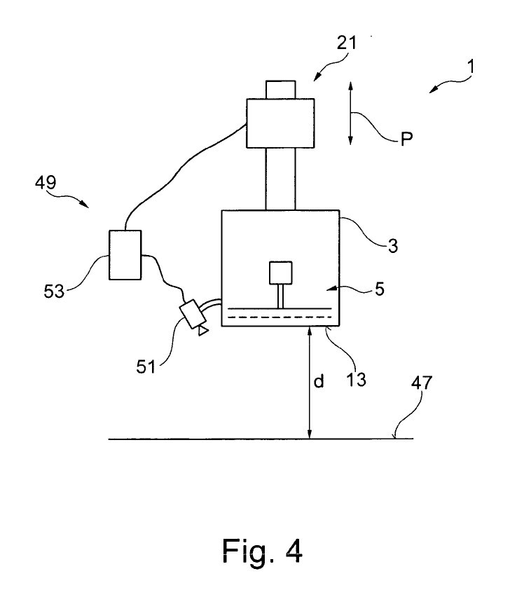

FIG. 4 shows a fourth practical example of an apparatus.

FIG. 1 shows a schematic view of an apparatus 1 for plasma treatment of surfaces, especially skin. It includes a base element, here a housing 3. A plasma source 5 through which a plasma can be generated for surface treatment is preferably integrated in it or provided on it.

The plasma source 5 can be designed differently:

A first preferred possibility proposes a plasma source according to the principle of dielectric barrier discharge (DBD). In this case an electrode is provided, which his acted upon with an alternating voltage, preferably a high frequency of alternative voltage. The electrode preferably includes a dielectric on its surface facing the surface being treated. The surface being treated serves as counterelectrode, in which case a plasma is ignited between the electrodes.

A plasma source including two electrodes, one of which is acted upon with an alternating voltage is also preferred, in which the other is preferably grounded. In order guarantee safety of a user, the grounded electrode is then preferably provided on a surface facing the surface being treated. The two electrodes are separated from each other by a dielectric. A discharge is ignited between the electrodes so that a plasma is formed on the side of the electrode facing the surface being treated, which acts on the surface being treated. This corresponds to the principle of surface microdischarge (SMD).

It is preferably also possible to provide a plasma source in which at least the electrode facing the surface being treated is embedded in a dielectric. It is then provided close enough to the surface of the dielectric that a plasma is formed on the surface when a discharge is ignited between the electrodes. The electrode is preferably also grounded in this case, which faces the surface being treated. An alternating voltage, with particular preference a high frequency alternating voltage is applied to the ungrounded electrode. The electrode not facing the surface being treated is also preferably embedded in a dielectric. In this case none of the electrodes is freely accessible. In particular, embedding of the electrode which faces the surface being treated corresponds in principle to a self-sterilizing surface (SSS).

In the practical examples depicted in the figures a plasma source is indicated schematically, which satisfies the principle of self-sterilizing surface. This corresponds to a preferred variant; it is likewise easily possible to provide a different plasma source.

In particular, if the apparatus 1 serves for treatment of skin, a plasma source 5 is preferably provided, which functions according to the principle of surface microdischarge or with particular preference a self-sterilizing surface. In this case exposure of skin to electric current is avoided so that plasma treatment is more comfortable and optionally also nonhazardous.

The plasma source 5 in the depicted practical example includes a first electrode 7 and a second electrode 9. Both electrodes 7, 9 are electrically connected to a voltage source 11 so that the first electrode 7 is exposed to a high voltage of appropriate frequency in which the second electrode 9 is preferably grounded. Both electrodes 7, 9 are preferably embedded in a dielectric, the second electrode 9 being arranged somewhat beneath a surface 13 in whose area a plasma is generated when a discharge is ignited between electrode 7, 9.

The apparatus preferably includes a switch 15, via which the plasma source 5 can be activated and deactivated. It also preferably includes a power supply 17, through which the voltage source 11 can be supplied power. The power supply 17, as shown here as an example, can be designed as a battery, storage battery or other power supply connected to housing 3. However, it is preferably also possible to provide a connection for an external power supply, for example, a plug. It is also possible to supply the plasma source 5 with power via an energy harvesting device. For example, piezocrystals, permanent magnets movable in coils, generally inductive devices, devices that are based on the principle of thermoelectricity, for example, the Seebeck effect, Peltier effect and/or Thomson effect, solar panels and numerous other known apparatuses are considered. These are preferably partly integrated in the voltage source 11 or replace it. For example, a piezocrystal can be provided, which simultaneously serves as voltage source and as power supply.

In particular, the power to generate the plasma is preferably taken from a movement of the apparatus 1 in which this can be a center of mass movement of apparatus 1, but also movement of parts of apparatus 1 relative to each other.

Apparatus 1 is preferably designed as a mobile, especially portable apparatus in particular as a hand-held device. In this case it preferably has a gripping area 19 in which a nonslip surface is preferably provided so that the user can securely grasp the apparatus 1.

In another practical example the apparatus 1 can preferably be designed as a fixed apparatus or in any event an apparatus not intended for simple transport. An apparatus provided in a treatment practice is conceivable, for example.

The apparatus 1 is particularly usable for treatment of wounds, skin irritation, infections, insect bites, fungal infestation of the feet, especially athlete's foot, acne, herpes, pimples, burns, infections, especially ear infections, rashes, especially diaper rash, heat blisters, fever blisters, lice infestation, especially lice infestation of the head or body, flea infestation, other infestation by invertebrates, dandruff, sensitive skin, fungal infestation of the nails and/or psoriasis. It can also preferably be used to prevent, eliminate, cover or mask, modify or reduce body odor. This includes foot odor, armpit odor, odor caused by incontinence, odor of the pubic area and/or odor escaping from the interior of the body.

In a preferred practical example the apparatus is provided for use in a warm, moist environment, for example, in a swimming pool or sauna. Here it is used preferably to avoid or treat foot fungus or other fungal infestation of the skin.

The apparatus is preferably also usable to reduce or prevent toothache.

It is then found that different chemical species are active or appropriate for different applications of the apparatus, which are enclosed by plasma or formed by it.

The term plasma then includes not only plasma in the narrower sense, i.e., the so-called “fourth state of aggregation”, which includes differently charged particles in a volume, which overall is quasi-neutral, but the term also includes uncharged reactive species formed by plasma in the narrower sense or together with it, for example, excited atoms or molecules, free radicals or other active species.

The apparatus 1 is suitable for application for cell regeneration. In this case nitrogen oxides (NO or NOₓ) are particularly effective against skin irritation.

The hydroxyl radial or OH radical is particularly effective for a bactericidal effect. It must be kept in mind here that this is toxic in high concentrations. A compromise between effective treatment and toxicity must therefore be made with reference to desired concentration in order to determine the concentration appropriate for treatment.

Ozone (O₃) is also particularly effective for the bactericidal effect of the plasma, but also to control undesired odors.

In this way different reactive species can be assigned to the different types of treatment, which preferably can be present in increased concentration at the treatment location.

Depending on the reaction rates with which these species form and decompose again and also depending on the specific transport mechanisms present, with which the plasma is transported from surface 13, on which it is generated, to the surface being treated, various distances are obtained from the surface 13 at which different species are present in increased concentration. Diffusion is considered in particular as transport mechanism. In this respect the diffusion constant of the reactants or the forming or decomposing species is relevant. Optionally, convection is also considered or a flow of the plasma is generated in certain practical examples, which must be then considered accordingly in selecting the treatment distance.

In any case, an almost location-dependent plasma chemistry can be stated so that an effectively performed treatment corresponds to a specific treatment distance, namely a specific distance from surface 13 to the surface being treated.

For example, reactive nitrogen species are present in particularly favorable concentration at a specific distance.

The distances relevant here typically lie on a length scale from 4 mm to 20 mm.

Undesired molecules on the surface being treated will optionally be dissociated or deactivated by the plasma in the narrower sense, including hot electrons, i.e., electrons with increased kinetic energy of preferably several electron volts. In this case a shorter distance of up to 2 mm can be chosen in which a sufficiently high concentration of hot electrons is present in order to deactivate or dissociate the undesired molecules. Such molecules preferably include odor molecules and/or odor-forming molecules that are metabolized, for example, by bacteria to odorous substances, or serve as a nutrient for bacteria, which again form odorous substances.

In order to be able to adjust the desired distance, the apparatus 1 includes a spacer device 21, by means of which a distance can be maintained between plasma source 5 and the surface being treated. This is designed adjustable so that the distance can be varied. A distance of about 0 to 30 mm, preferably about 4 to 20 mm, is adjustable. This ensures that an appropriate plasma chemistry for the desired type of treatment can be selected. If a distance of 2 mm or less can be adjusted, hot electrons can also be selected for deactivation of undesired molecules on the surface being treated.

In order to produce a variation of the treatment distance, the spacer device 21 in the depicted practical example includes an internal thread 23, which meshes with an external thread 25, which is provided on housing 3. The spacer device 21 and the housing 3 in this case can be rotated relative to each other in order to adjust the treatment distance.

In another practical example the spacer device 21 includes at least one snap element that cooperate with at least a corresponding snap element of the apparatus, especially a snap element provided on the housing 3 in order to define the distance.

In another practical example it is possible that the spacer device 21 can be moved relative to housing 3 viewed in the longitudinal direction of apparatus 1 in which case the friction conditions between the spacer device 21 and the housing 3 are chosen so that a specific distance can be reliably adjusted by feel without requiring a defined snap-in position. The distance is then continuously variable.

With particular preference apparatus 1 has a scale by means of which a desired distance or a preferred plasma chemistry can be adjusted. In the depicted practical example a scale 27 is provided from the spacer device 21. The scale 27 can show the treatment distance, a table preferably being provided in this case which assigns a desired type of treatment or the plasma chemistry matching it to a specified distance. However, it is also possible to provide pictograms instead of a numerical scale 27, from which desired treatment types or a specific plasma chemistry follow.

The plasma generated by means of plasma source 5 has the property of being able to enter the fabric of textile materials or penetrating textile materials. Application of apparatus 1 through clothing is therefore also possible. However, in this case the distance caused by the clothing must be added to the distance stipulated by the spacer device 21.

It is found during treatment of textile surfaces that the plasma has a certain depth effect owing to its capability to penetrate into the fabric. The term “surface” therefore includes in this case a certain co-treated volume in the textile material. It is also found in the treatment of skin that a certain depth effect exists owing to the fact that paracrine effects or intercellular communication are involved in the effect mechanism. A certain treatment effect is then almost conveyed from cell to cell so that it is also effective beyond the actually treated surface.

The apparatus 1 preferably includes an application device 29 through which at least one additive can be applied to the surface being treated. The application device 29 in the depicted practical example is designed as a spray, in which case it includes a nozzle 31, feed line 33 and a supply container 35. The nozzle 31 is connected to supply container 35 via feed line 33. It is preferably refillable and/or replaceable in housing 3. A pump device is not shown, via which the one additive is preferably transported from the supply container 35 via feed line 33 to nozzle 31 and delivered from it. The pump device can be operated automatically, especially electrically or also manually, especially in the fashion of a pump spray. It is the possible to activate the pump device before, after or simultaneously with plasma source 5. A spray mist 37 discharged from nozzle 31 is depicted schematically.

In another practical example the application device can be designed as a roller device, for example, in the fashion of a deodorant roller, or as a stick or pencil, especially in the fashion of a deodorant pencil or stick.

A substance that removes, modifies, masks, reduces an odor on the surface being treated or prevents its formation is preferably considered as additive. To this extent the additive can include a perfume or deodorant. It can also include an antiperspirant, which prevents sweat formation. It is possible at the same time that the additive covers a “technical” odor of the plasma.

In another preferred practical example, the additive is chosen so that it supports the bactericidal effect of the plasma, inhibits bacterial growth, has a disinfecting effect, acts antivirally and/or has decontaminating properties with respect to the surface being treated.

Finally, the additive preferably includes a substance that is suitable for long treatment, alleviates skin irritations, is suitable for treatment of insect bites, serves for fungal treatment, especially is fungicidal, suitable for treatment of acne, herpes or pimples, is suitable for treatment of burns, alleviates ear infections or controls them, is suitable to alleviate or control rash, especially diaper rash, is suitable for treatment of heat blisters, fever blisters or other blistering of the skin, suitable to control lice and/or fleas or other invertebrates, has an antidandruff property, is suitable for treatment of sensitive skin, prevents, alleviates or controls dandruff, promotes or inhibits hair growth, has a decorating effect and/or has other properties that are desirable in conjunction with the surface being treated, especially in conjunction with treatment of skin. The additive then preferably supports the effect achieved with the chosen type of treatment.

It is naturally possible to use different additives in combination with each other, in succession, as an alternative to each other and/or in any mixture.

A cold plasma is preferably formed by the plasma source 5 so that the surface being treated, especially skin, is not exposed to high thermal load. Little UV radiation is also generated with particular preference during plasma generation so that the surface being treated is not excessively loaded by this. In many types of treatment, however, irradiation with UV radiation can be desirable. In this case it is proposed that the UV intensity of plasma source 5 is preferably increased, especially adapted to a desired intensity.

FIG. 2 shows a schematic view of a second practical example of the apparatus. Identical and functionally identical elements are provided with the same reference numbers so that the previous description is referred to in this respect.

Surface 13 is designed curved here, especially convex. A curved surface 13 has the advantage that it can be adapted in particular to the curvature of a surface being treated. Even in irregularly curved surfaces being treated there is always at least one point at which a desired treatment distance is present.

Depending on the curvature of the surface being treated, on the one hand, and the curvature of the surface 13, on the other, there is an entire family of treatment distances so that different effect mechanisms can be accomplished simultaneously. For example, if in the practical example depicted in FIG. 2 the highest point of surface 13 approaches the surface being treated so closely that dissociation of molecules from hot electrons occurs in this area, radially farther out there is an area in which the bactericidal effect of the plasma predominates. At another radial distance to the highest point of surface 13 the regenerative effect of the on skin cells predominates or the effect against skin irritation. It is therefore found that a curved surface (be it convex, concave or irregularly curved) can simultaneously include a number of possible effect mechanisms. If the surface being treated is then covered by the apparatus 1, each of the treatment mechanisms can be effective on each point.

In the practical example depicted in FIG. 2 a very simple spacer device 21 is provided. This includes here merely a rod 39, which is arranged movable on the housing 3 preferably by means of a clamping device 41. Ultimately, rod 39 is sufficient to guarantee a defined distance to the highest point of surface 13. However, a spacer ring 43 is preferably provided on rod 39, which is preferably positioned on the surface being treated in order to define the treatment distance.

FIG. 3 shows a schematic view of a third practical example of apparatus 1. Identical and functionally identical elements are provided with the same reference numbers so that the previously description is referred to in this respect. Surface 13 is designed flexible and/or elastic here and is preferably connected to the spacer device 21 so that it can be moved together with it and deformed by it. For this purpose the surface 13 with particular preference is fastened to housing 3 at its lowest point. If the spacer device 21 is moved, the surface 13 is deformed and then changes in particular its curvature. The electrodes 7, 9 are preferably integrated in the surface 13.

Surface 13 is designed concave here. If a surface being treated is positioned on an upper edge 45 of the spacer device 21, a closed volume is produced in which the plasma is formed. This is particular advantageous because no disturbances from the outside act on the plasma, for example, no air currents. Particularly intensive plasma treatment is thus possible. The power of the plasma source 5 can optionally also be reduced, which is particularly advantageous when sensitive skin is treated. As already discussed in conjunction with FIG. 2, different effect mechanisms are produced in different areas of surface 13 in a concave surface 13, depending on the curvature of the surface being treated.

Deviating from the depiction according to FIG. 3, as an alternative or in addition it is possible to raise and/or lower the lowest point of the flexible or elastic surface 13 relative to housing 3. For this purpose a type of punch or another appropriate actuator can be provided. Since the lowest point of the surface 13 can be varied in this way relative to its distance to a surface being treated, it is not absolutely necessary that the spacer device 21 be designed variable. In contrast, the adjustable lowest point is again achieved with a fixed spacer device 21 in which a treatment distance between the lowest point and the surface being treated can be variably defined.

The surface 13 preferably includes a rubber-like elastic material, with particular preference rubber. The electrodes 7, 9 preferably integrated in the flexible and/or elastic material are also themselves flexible and/or elastic with particular preference.

FIG. 4 shows a schematic view of a fourth practical example of apparatus 1. Identical and functionally identical elements are provided with the reference numbers so that the previous description is referred to in this respect. Apparatus 1 is designed here as a nontransportable, preferably fixed apparatus. All features and functions described below in conjunction with the apparatus 1 depicted here, however, are also provided in other preferred practical examples in mobile, portable apparatuses, especially in handheld devices.

Apparatus 1 serves for treatment of a surface 47, in which case the distance between surface 13 and surface 47, i.e., the treatment distance, is marked d.

A spacer device 21 is provided by means of which housing 3, which includes the plasma source 5, can be moved in the direction of arrow P to surface 47 or away from it in order to adjust a desired treatment distance d.

It is essential in the depicted practical example of apparatus 1 that it includes a selection device 49 by means of which the treatment distance d can be selected.

For this purpose selection device 49 preferably includes a sensor, here a camera 51, to record properties of the surface 47 being treated. In other preferred practical examples the sensor is designed as a measurement sensor, for example, as a resistance measurement device or also “sniffer”, for example, as a gas chromatograph or ion mobility measurement cell, in which case it preferably draws in molecules or other particles arranged on the surface 47 and can determine their identity. In case the characteristic value of surface 47 can be recorded with the sensor.

The sensor, here camera 51, is preferably connected to a comparison device 53, for example, a computer. This evaluates the data coming from the sensor with respect to properties of surface 47 and a treatment type to be selected according to these properties. It is preferably linked to a specific plasma chemistry, which is determined by the comparison device 53. The comparison device 53 preferably calculates the distance d, which is suitable with respect to the selected plasma chemistry. It is also possible that a specified distance d is simultaneously linked with a treatment type chosen by the comparison device 53 without requiring calculation of the distance based on plasma chemistry.

Evaluation of the data occurs in comparison device 23 preferably by comparison of at least one recorded value with at least one reference value, preferably a number of reference values. These are preferably characteristic for certain states of the surface being treated, which require a certain treatment type. The comparison device 53 assigns at least one reference value to the recorded value so that a specified state of the surface being treated is identified and a treatment type established. A desired plasma chemistry can be linked to this, by means of which the distance d can be calculated. In another practical example a distance d is directly assigned to each reference value so that it need not be calculated first.

The comparison device 53 is preferably connected to the spacer device 21 so that the selected or calculated distance d can be automatically adjusted.

In another practical example it is possible that the comparison device 53 displays the chosen or calculated distance d so that it could be adjusted manually by means of the spacer device 21.

If the type of treatment, as in the practical example depicted in FIG. 4, is determined optically by means of a camera 51, the following method steps are preferably conducted:

Camera 51 records at least one picture of surface 47. This is compared in the comparison device 53 with at least one reference image, preferably with a family of reference images stored in a database. As a result of this comparison, at least matching reference image is assigned to the image recorded by the camera 51. For example, such a reference image shows an insect bite, skin irritation or other properties of surface 47 requiring treatment.

Treatment types, distances d and/or parameters of a plasma chemistry that are optimal for treatment of the interpretation of surface 47 depicted on the corresponding reference image are preferably linked to the reference images filed in the database.

If a distance d is directly linked to the reference image, this can be chosen directly and preferably sent to the spacer device 21. As an alternative a corresponding signal can be sent to the spacer device 21 to adjust the chosen distance.

If a treatment type is linked to the reference image, this is again linked either to a specific plasma chemistry or directly to a specific preferred distance d.

Finally, if the reference image is linked to a certain plasma chemistry, this is again linked either to a specified distance or the distance is preferably calculated, depending on the desired plasma chemistry by means of at least one reaction rate for formation and/or decomposition of a desired species as well as at least one relevant diffusion constant for the plasma.

It goes without saying that the possibilities or variants of the method discussed here with respect to determination of distance d from comparison of the recorded images with reference images are just as applicable if other characteristic values are compared with corresponding reference values instead of images.

It is also possible with particular preference by means of selection device 49 to select the treatment intensity, i.e., the concentration of plasma acting on surface 47 and/or the treatment time with reference to data obtained about surface 47.

It is also possible in conjunction with evaluation device 49 to combine several sensors with each other, for example, a camera 51 and a measurement sensor. A desired treatment type can be selected even more precisely on this account.

Data of a plasma chemistry desired for treatment is with particular preference linked to the reference data because this is independent of the specific embodiment of apparatus 1. A corresponding database can then be used by a number of apparatuses 1.

On the other hand, a specific distance d is specific for a specific apparatus 1, because specific transport mechanisms from surface 13 to surface 47, the intensity of the plasma source 5 and other parameters of the apparatus 1 enter into this distance.

Optical diagnosis of surface 47 is preferred because this occurs without contact and very quickly.

It is essential for the method for plasma treatment of surfaces, especially skin, that an effect mechanism be chosen for the surface being treated so that a distance from the plasma source of the surface being treated is adjusted and plasma treatment is conducted.

Finally, features of the method also became clear based on a description of the properties of the practical examples of apparatus 1 presented here.

Overall it is found that it is possible by means of the apparatus, the method and use of the apparatus to conduct different plasma treatments of surfaces by means of the same apparatus, especially by means of a mobile device portable in the hand, in which case an optimal plasma chemistry for the desired type of treatment can be adjusted.

1. An apparatus for plasma treatment of surfaces, said apparatus comprising:
a housing;
a plasma source connected to the housing and
at least one spacer device, which is provided on the housing and/or plasma source so that a distance can be maintained between the plasma source and a surface being treated at least in areas,
wherein
the spacer device is adjustable in order to vary the distance and
the distance is variable so that a preferred plasma chemistry can be selected at a location of the surface being treated.

2. The apparatus according to claim 1, wherein the spacer device is sufficiently adjustable such that the distance can be varied from about 1 to 30 mm.

3. The apparatus according to claim 1, wherein the spacer device has a thread that meshes with a corresponding thread of the apparatus.

4. The apparatus according to claim 1, wherein the spacer device has at least one snap element that cooperates with at least one corresponding snap element of the apparatus in order to define the distance.

5. The apparatus according to claim 1, further comprising a scale by way of which a desired distance or a preferred plasma chemistry can be adjusted.

6. The apparatus according to claim 1, wherein the plasma source includes a service in whose area the plasma is formed, in which the surface is curved.

7. The apparatus according to further comprising an application device through which at least one additive can be applied to the surface being treated.

8. The apparatus according to claim 7, wherein the application device is a spray device, roller device or stick device.

9. The apparatus according to claim 1, further comprising a selection device for automatic selection of a preferred plasma chemistry.

10. A method for plasma treatment of surfaces by use of an apparatus according to claim 1, said method comprising the following steps:
selection of an effect mechanism for the surface being treated;
adjustment of a distance from the plasma source to the surface being treated and
performance of plasma treatment.

11. The method according to claim 10, wherein the distance is set from about 0 to 30 mm.

12. The method according to claim 10, wherein a selection of the distance is conducted automatically by a selection device, in which the following steps are performed:
detection of at least one characteristic value of the surface being treated;
comparison of the at least one characteristic value with at least one reference value;
assignment of the at least one characteristic value to the at least one reference value; and
determination of the distance by use of the at least one assigned reference value.

13. The method according to claim 12, comprising the following steps:
recording of a picture of the surface being treated;
comparison of the picture with at least one reference picture;
assignment of the picture to the at least one reference picture;
selection of a plasma chemistry preferably linked to the at least one reference picture, and
determination of a distance which corresponds to the chosen plasma chemistry.

14. The method according to claim 10, wherein the distance is calculated as a function of a desired plasma chemistry by use of at least one reaction rate for formation and/or decomposition of a desired species as well as at least one relevant diffusion constant for the plasma.

15. The method according to claim 10, further comprising applying at least one additive to the surface being treated.

16. The method according to claim 10, wherein the surfaces comprise skin and the method treats wounds, skin irritation, infections, insect bites, fungal infestation of the feet, acne, herpes, pimples, burns, ear infections, rashes, heat blisters, lice infestation, flea infestation, other infestation by invertebrates, dandruff, sensitive skin, fungal infestation of the nails, psoriasis, fever blisters, and/or the method prevents, removes, modifies or reduces body odor.
